(19) United States
(12) Patent Application Publication
(10) Pub. No.: US 2017/0079556 A1
(43) Pub. Date: Mar. 23, 2017

(54) NITRIC OXIDE CONCENTRATION TESTING DEVICE, NITRIC OXIDE CONCENTRATION TESTING SYSTEM, NITRIC OXIDE CONCENTRATION TESTING SERVER, AND NITRIC OXIDE CONCENTRATION TESTING TERMINAL DEVICE

(71) Applicant: Seiko Epson Corporation, Tokyo (JP)

(72) Inventor: Yoshifumi HANO, Kai (JP)

(21) Appl. No.: 15/254,076

(22) Filed: Sep. 1, 2016

(30) Foreign Application Priority Data
Sep. 18, 2015 (JP) 2015-184811

(51) Int. Cl. A61B 5/08; A61B 5/00; A61M 15/00

(52) U.S. Cl. CPC A61B 5/082; A61M 15/00; A61B 5/7221; A61B 5/0075; A61B 5/0002

(57) ABSTRACT

A nitric oxide concentration testing device includes a detection section which detects a nitric oxide concentration in the breath of a test subject, an acquisition section which acquires use history information which is information on the use history of a drug inhalation device, and a memory section which stores nitric oxide concentration information which is information on the nitric oxide concentration, measurement time information which is information on the measurement time of the nitric oxide concentration, and the use history information.

### BACKGROUND

1. Technical Field

The present invention relates to a nitric oxide concentration testing device, a nitric oxide concentration testing system, a nitric oxide concentration testing server, and a nitric oxide concentration testing terminal device.

2. Related Art

Recently, asthma has begun to be regarded as basically airway inflammatory reaction. Therefore, as a therapeutic method for asthma, drug therapy represented by inhaled corticosteroid has been recommended. As an index of airway inflammatory conditions, nitric oxide in the breath has attracted attention. The amount of nitric oxide (NO) in the breath correlates with the degree of airway inflammation, and by monitoring the amount of nitric oxide in a certain period of time, the degree of airway inflammation can be monitored. For example, JP-A-2012-18182 (PTL 1) discloses a device which detects the amount nitric oxide in the breath.

However, the degree of airway inflammation may be improved temporarily immediately after a drug such as inhaled corticosteroid is inhaled. Due to this, when a nitric oxide concentration in the breath is measured immediately after a drug such as inhaled corticosteroid is inhaled, the nitric oxide concentration which correctly reflects the true degree of airway inflammation cannot be measured, and therefore, highly reliable information may not be able to be obtained.

### SUMMARY

An advantage of some aspects of the invention is to provide a nitric oxide concentration testing device, a nitric oxide concentration testing system, a nitric oxide concentration testing server, a nitric oxide concentration testing terminal device, and the like, each capable of obtaining highly reliable information.

The invention can be implemented as the following forms or application examples.

### APPLICATION EXAMPLE 1

A nitric oxide concentration testing device according to this application example includes: a detection section which detects a nitric oxide concentration in the breath of a test subject; an acquisition section which acquires use history information which is information on the use history of a drug inhalation device; and a memory section which stores nitric oxide concentration information which is information on the nitric oxide concentration, measurement time information which is information on the measurement time of the nitric oxide concentration, and the use history information.

According to this application example, a chronological relationship between the use time of the drug inhalation device and the measurement time of the nitric oxide concentration can be ascertained, and therefore, it becomes easy to determine which information is highly reliable nitric oxide concentration information. Accordingly, a nitric oxide concentration testing device capable of obtaining highly reliable information can be realized.

### APPLICATION EXAMPLE 2

In the nitric oxide concentration testing device according to the application example, it is preferred that the device includes a determination section which determines the validity of the nitric oxide concentration information based on information on the use time of the drug inhalation device obtained from the use history information and the measurement time information.

According to this application example, it is determined which information is highly reliable nitric oxide concentration information based on the use time of the drug inhalation device and the measurement time of the nitric oxide concentration, and therefore, a nitric oxide concentration testing device capable of obtaining highly reliable information can be realized.

### APPLICATION EXAMPLE 3

In the nitric oxide concentration testing device according to the application example, it is preferred that the device includes a time determination section which determines the use time of the drug inhalation device based on the use history information.

According to this application example, a nitric oxide concentration testing device capable of obtaining highly reliable information can be realized even using a drug inhalation device with a simple structure.

### APPLICATION EXAMPLE 4

In the nitric oxide concentration testing device according to the application example, it is preferred that the use history information includes information on the movement of the drug inhalation device.

The information on the movement of the drug inhalation device may be, for example, information on a time and an acceleration rate.

According to this application example, a nitric oxide concentration testing device capable of obtaining highly reliable information can be realized even using a drug inhalation device with a simple structure.

### APPLICATION EXAMPLE 5

In the nitric oxide concentration testing device according to the application example, it is preferred that the use history information includes information on the operation of the drug inhalation device.

The information on the operation of the drug inhalation device may include information on whether or not a specific operation section of the drug inhalation device is operated and information on a time when the operation section is operated.

According to this application example, a nitric oxide concentration testing device capable of obtaining highly reliable information can be realized even using a drug inhalation device with a simple structure.

### APPLICATION EXAMPLE 6

In the nitric oxide concentration testing device according to the application example, it is preferred that the detection section detects the nitric oxide concentration using spectroscopy.

According to this application example, the nitric oxide concentration is detected using spectroscopy, and therefore, a nitric oxide concentration testing device which has excellent measurement reliability can be realized.

### APPLICATION EXAMPLE 7

In the nitric oxide concentration testing device according to the application example, it is preferred that the detection section detects the nitric oxide concentration using a surface-enhanced Raman scattering method.

According to this application example, the nitric oxide concentration is detected using a surface-enhanced Raman scattering method, and therefore, a nitric oxide concentration testing device which is less likely to be affected by the environment, and is highly sensitive and small in size can be realized.

### APPLICATION EXAMPLE 8

A nitric oxide concentration testing system according to this application example includes: a detection section which detects a nitric oxide concentration in the breath of a test subject; an acquisition section which acquires use history information which is information on the use history of a drug inhalation device; and a memory section which stores nitric oxide concentration information which is information on the nitric oxide concentration, measurement time information which is information on the measurement time of the nitric oxide concentration, and the use history information in association with one another.

According to this application example, a chronological relationship between the use time of the drug inhalation device and the measurement time of the nitric oxide concentration can be ascertained, and therefore, it becomes easy to determine which information is highly reliable nitric oxide concentration information. Accordingly, a nitric oxide concentration testing system capable of obtaining highly reliable information can be realized.

### APPLICATION EXAMPLE 9

A nitric oxide concentration testing server according to this application example is includes: a receiving section which receives nitric oxide concentration information which is information on a nitric oxide concentration in the breath of a test subject, measurement time information which is information on the measurement time of the nitric oxide concentration, and use history information which is information on the use history of a drug inhalation device from a nitric oxide concentration testing terminal including a detection section which detects the nitric oxide concentration and an acquisition section which acquires the use history information; and a memory section which stores the nitric oxide concentration information, the measurement time information, and the use history information.

According to this application example, a chronological relationship between the use time of the drug inhalation device and the measurement time of the nitric oxide concentration can be ascertained, and therefore, it becomes easy to determine which information is highly reliable nitric oxide concentration information. Accordingly, a nitric oxide concentration testing server capable of obtaining highly reliable information can be realized.

### APPLICATION EXAMPLE 10

A nitric oxide concentration testing terminal device according to this application example includes: a detection section which detects a nitric oxide concentration in the breath of a test subject; an acquisition section which acquires use history information which is information on the use history of a drug inhalation device; and a transmission section which transmits nitric oxide concentration information which is information on the nitric oxide concentration, measurement time information which is information on the measurement time of the nitric oxide concentration, and the use history information to a nitric oxide concentration testing server including a memory section which stores the nitric oxide concentration information, the measurement time information, and the use history information in association with one another.

According to this application example, a chronological relationship between the use time of the drug inhalation device and the measurement time of the nitric oxide concentration can be ascertained, and therefore, it becomes easy to determine which information is highly reliable nitric oxide concentration information. Accordingly, a nitric oxide concentration testing terminal device capable of obtaining highly reliable information can be realized.

### APPLICATION EXAMPLE 11

A nitric oxide concentration testing server according to this application example includes: a receiving section which receives nitric oxide concentration information which is information on a nitric oxide concentration in the breath of a test subject and measurement time information which is information on the measurement time of the nitric oxide concentration from a nitric oxide concentration testing terminal including a detection section which detects the nitric oxide concentration; an acquisition section which acquires use history information which is information on the use history of a drug inhalation device; and a memory section which stores the nitric oxide concentration information, the measurement time information, and the use history information.

According to this application example, a chronological relationship between the use time of the drug inhalation device and the measurement time of the nitric oxide concentration can be ascertained, and therefore, it becomes easy to determine which information is highly reliable nitric oxide concentration information. Accordingly, a nitric oxide concentration testing server capable of obtaining highly reliable information can be realized.

### APPLICATION EXAMPLE 12

A nitric oxide concentration testing terminal device according to this application example includes: a detection section which detects a nitric oxide concentration in the breath of a test subject; and a transmission section which transmits nitric oxide concentration information which is information on the nitric oxide concentration and measurement time information which is information on the measurement time of the nitric oxide concentration to a nitric oxide concentration testing server including a memory section which stores the nitric oxide concentration information, the measurement time information, and use history information which is information on the use history of a drug inhalation device in association with one another.

According to this application example, a chronological relationship between the use time of the drug inhalation device and the measurement time of the nitric oxide concentration can be ascertained, and therefore, it becomes easy to determine which information is highly reliable nitric oxide concentration information. Accordingly, a nitric oxide concentration testing terminal device capable of obtaining highly reliable information can be realized.

### BRIEF DESCRIPTION OF THE DRAWINGS

The invention will be described with reference to the accompanying drawings, wherein like numbers reference like elements.

FIG. 1 is a view schematically showing a use mode of a nitric oxide concentration testing device 1 according to an embodiment.

FIG. 2 is a functional block diagram of the nitric oxide concentration testing device 1 according to the embodiment.

FIG. 3 is a table showing an example of information stored in a memory section 13.

FIG. 4 is a view showing an overall configuration of a nitric oxide concentration testing system 2 according to a first embodiment.

FIG. 5 is a functional block diagram of a nitric oxide concentration testing terminal device 3 according to the first embodiment.

FIG. 6 is a functional block diagram of a nitric oxide concentration testing server 4 according to the first embodiment.

FIG. 7 is a view showing an overall configuration of a nitric oxide concentration testing system 2a according to a second embodiment.

FIG. 8 is a functional block diagram of a nitric oxide concentration testing terminal device 3a according to the second embodiment.

FIG. 9 is a functional block diagram of a nitric oxide concentration testing server 4a according to the second embodiment.

FIG. 10 is a plan view schematically showing a sensor chip 300.

FIG. 11 is a cross-sectional view taken along the line V-V in FIG. 10 schematically showing the sensor chip 300.

FIG. 12 is a view conceptually showing the principle of trapping nitric oxide.

### DESCRIPTION OF EXEMPLARY EMBODIMENTS

Hereinafter, preferred embodiments of the invention will be described in detail with reference to the drawings. The drawings to be used are for reference only to facilitate the explanation. The embodiments described below do not unduly limit the contents of the invention described in the appended claims. Also, all of the configurations described below are not necessarily essential components of the invention.

### 1. Nitric Oxide Concentration Testing Device

### 1-1. Overall Configuration

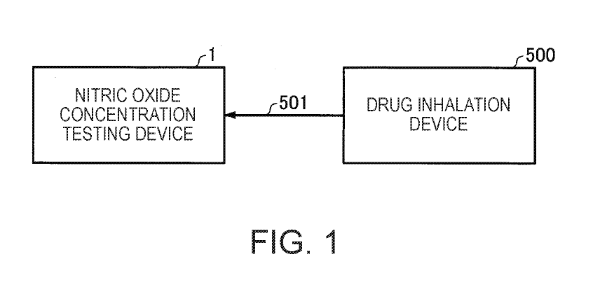

FIG. 1 is a view schematically showing a use mode of a nitric oxide concentration testing device 1 according to this embodiment.

The nitric oxide concentration testing device 1 according to this embodiment acquires use history information 501 which is information on the use history of a drug inhalation device from a drug inhalation device 500.

The drug inhalation device 500 is a device for a patient to inhale a drug such as inhaled corticosteroid to be used for a treatment of asthma, COPD (chronic obstructive pulmonary disease), or the like.

### 1-2. Configuration of Nitric Oxide Concentration Testing Device

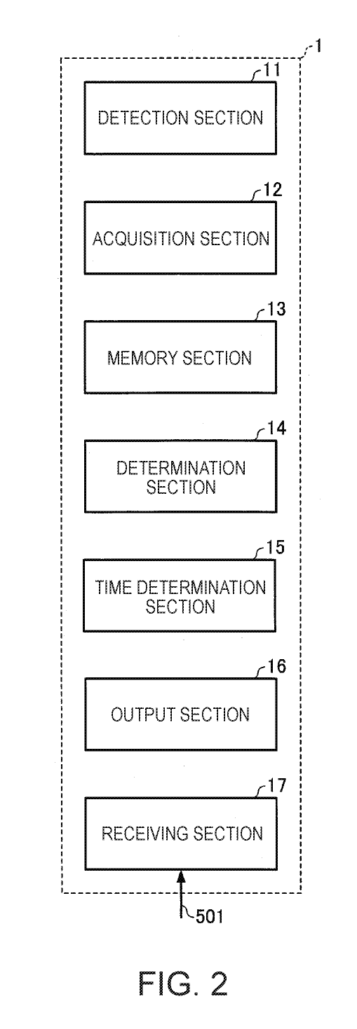

FIG. 2 is a functional block diagram of the nitric oxide concentration testing device 1 according to this embodiment. In an example shown in FIG. 2, the nitric oxide concentration testing device 1 is configured to include a detection section 11, an acquisition section 12, a memory section 13, a determination section 14, a time determination section 15, an output section 16, and a receiving section 17. Some components shown in FIG. 2 may be omitted, or another component may be added according to a function required for the nitric oxide concentration testing device 1.

The detection section 11 detects a nitric oxide concentration in the breath of a test subject. A detailed configuration example of the detection section 11 will be described later in the item of “3. Configuration Example of Detection Section”.

The acquisition section 12 acquires the use history information 501 which is information on the use history of the drug inhalation device 500. In this embodiment, the acquisition section 12 acquires the use history information 501 through the receiving section 17. The use history information 501 may include, for example, information on the time when a test subject (patient) used the drug inhalation device 500.

The memory section 13 stores nitric oxide concentration information which is information on a nitric oxide concentration in the breath of a test subject, measurement time information which is information on the measurement time of a nitric oxide concentration in the breath of a test subject, and the use history information 501 which is information on the use history of the drug inhalation device 500. In this embodiment, the memory section 13 stores the nitric oxide concentration information which is information on a nitric oxide concentration in the breath of a test subject and the measurement time information which is information on the measurement time of a nitric oxide concentration in the breath of a test subject in association with each other.

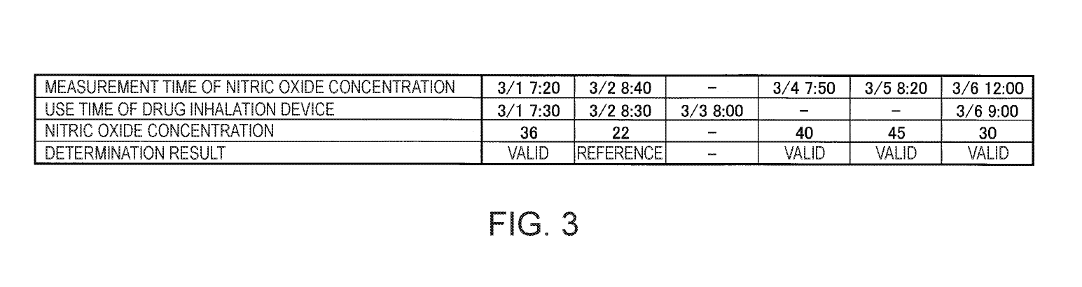

FIG. 3 is a table showing an example of information stored in the memory section 13. The first line in FIG. 3 shows the measurement time of the nitric oxide concentration. This information corresponds to the measurement time information. The second line in FIG. 3 shows the use time of the drug inhalation device 500. This information corresponds to the use history information 501. The third line in FIG. 3 shows the nitric oxide concentration (ppb). This information corresponds to the nitric oxide concentration information. In the example shown in FIG. 3, information corresponding to the same date is shown in the same column.

According to this embodiment, a chronological relationship between the use time of the drug inhalation device 500 and the measurement time of the nitric oxide concentration can be ascertained, and therefore, it becomes easy to determine which information is highly reliable nitric oxide concentration information. For example, in the case where the nitric oxide concentration is measured outside the period of 2 hours after the drug inhalation device 500 is used, a doctor may determine that the reliability of the corresponding nitric oxide concentration information is high (valid data). Further, for example, in the case where the nitric oxide concentration is measured within the period of 2 hours after the drug inhalation device 500 is used, a doctor may determine that the reliability of the corresponding nitric oxide concentration information is low. Accordingly, the nitric oxide concentration testing device 1 capable of obtaining highly reliable information can be realized.

With reference back to FIG. 2, the determination section 14 determines the validity of the nitric oxide concentration information which is information on a nitric oxide concentration in the breath of a test subject based on information on the use time of the drug inhalation device 500 obtained from the use history information 501 of the drug inhalation device 500 and the measurement time information which is information on the measurement time of a nitric oxide concentration in the breath of a test subject. For example, in the case where the nitric oxide concentration is measured outside the period of 2 hours after the drug inhalation device 500 is used, the determination section 14 may determine that the reliability of the corresponding nitric oxide concentration information is high (valid data). Further, for example, in the case where the nitric oxide concentration is measured within the period of 2 hours after the drug inhalation device 500 is used, the determination section 14 may determine that the reliability of the corresponding nitric oxide concentration information is low (reference data).

The determination section 14 may store the determination result in the memory section 13. The memory section 13 stores the nitric oxide concentration information which is information on a nitric oxide concentration in the breath of a test subject, the measurement time information which is information on the measurement time of a nitric oxide concentration in the breath of a test subject, the use history information 501 which is information on the use history of the drug inhalation device 500, and the determination result by the determination section 14 in association with one another. For example, the fourth line in FIG. 3 shows the determination result by the determination section 14.

According to this embodiment, it is determined which information is highly reliable nitric oxide concentration information based on the use time of the drug inhalation device 500 and the measurement time of the nitric oxide concentration, and therefore, the nitric oxide concentration testing device 1 capable of obtaining highly reliable information can be realized.

The time determination section 15 determines the use time of the drug inhalation device 500 based on the use history information 501 of the drug inhalation device 500.

For example, the use history information 501 of the drug inhalation device 500 may include information on the movement of the drug inhalation device 500. The information on the movement of the drug inhalation device 500 may be, for example, information on a time and an acceleration rate. The information on the movement of the drug inhalation device 500 may be obtained from, for example, an acceleration rate sensor provided in the drug inhalation device 500.

Further, for example, the use history information 501 of the drug inhalation device 500 may include information on the operation of the drug inhalation device 500. The information on the operation of the drug inhalation device 500 may include information on whether or not a specific operation section (for example, an operation button for inhaling a drug) of the drug inhalation device 500 is operated and information on a time when the operation section is operated.

According to this embodiment, the nitric oxide concentration testing device 1 capable of obtaining highly reliable information can be realized even using a drug inhalation device 500 with a simple structure.

The output section 16 outputs information stored in the memory section 13. The output section 16 may output information stored in the memory section 13 to, for example, a portable external memory device. Further, for example, the output section 16 may output information stored in the memory section 13 to a display section (not shown) provided in the nitric oxide concentration testing device 1. Further, for example, the output section 16 may output information stored in the memory section 13 to an external server through a communication network (not shown).

The receiving section 17 receives the use history information 501 of the drug inhalation device 500. The receiving section 17 receives the use history information 501 from the drug inhalation device 500 using at least one of wired communication and wireless communication.

### 2. Nitric Oxide Concentration Testing System, Nitric Oxide Concentration Testing Server, and Nitric Oxide Concentration Testing Terminal Device

### 2-1. First Embodiment

### 2-1-1. Overall Configuration

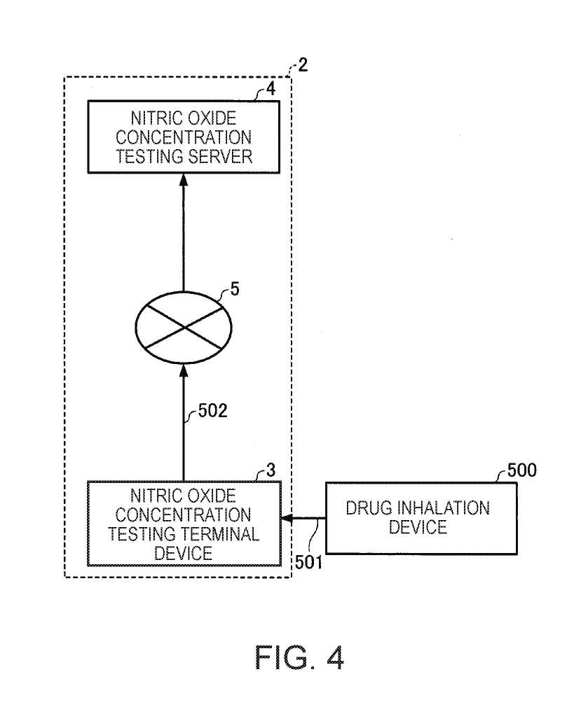

FIG. 4 is a view showing an overall configuration of a nitric oxide concentration testing system 2 according to a first embodiment. The same reference numerals are given to the same components as those in FIG. 1, and a detailed description thereof will be omitted.

The nitric oxide concentration testing system 2 shown in FIG. 4 is configured to include a nitric oxide concentration testing terminal device 3 and a nitric oxide concentration testing server 4.

The nitric oxide concentration testing terminal device 3 acquires use history information 501 which is information on the use history of a drug inhalation device from a drug inhalation device 500. The nitric oxide concentration testing terminal device 3 transmits information 502 (the content of the information 502 will be described later) to the nitric oxide concentration testing server 4 through a communication network 5.

As described below, according to this embodiment, a chronological relationship between the use time of the drug inhalation device 500 and the measurement time of the nitric oxide concentration can be ascertained, and therefore, it becomes easy to determine which information is highly reliable nitric oxide concentration information. Accordingly, the nitric oxide concentration testing system 2 capable of obtaining highly reliable information can be realized.

### 2-1-2. Configuration of Nitric Oxide Concentration Testing Terminal Device

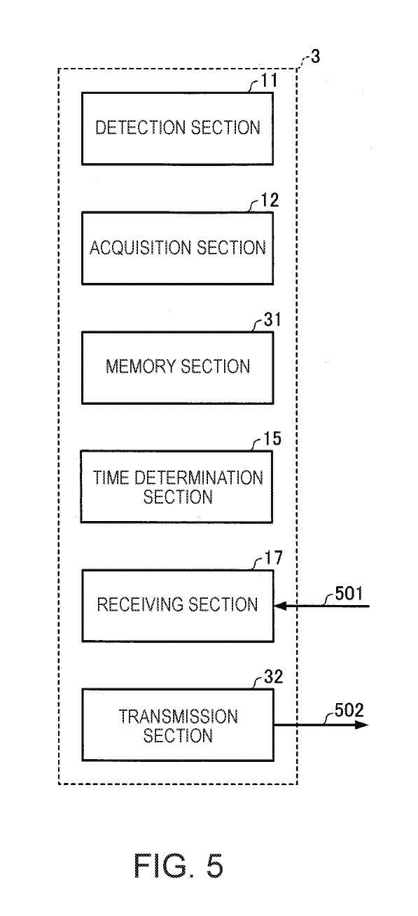

FIG. 5 is a functional block diagram of the nitric oxide concentration testing terminal device 3 according to the first embodiment. The same reference numerals are given to the same components as those in FIG. 2, and a detailed description thereof will be omitted.

In an example shown in FIG. 5, the nitric oxide concentration testing terminal device 3 is configured to include a detection section 11, an acquisition section 12, a memory section 31, a time determination section 15, a receiving section 17, and a transmission section 32. Some components shown in FIG. 5 may be omitted, or another component may be added according to a function required for the nitric oxide concentration testing terminal device 3.

The memory section 31 stores nitric oxide concentration information which is information on a nitric oxide concentration in the breath of a test subject, measurement time information which is information on the measurement time of a nitric oxide concentration in the breath of a test subject, and the use history information 501 which is information on the use history of the drug inhalation device 500 in association with one another. The example of the information stored in the memory section 31 is the same as described with reference to FIG. 3.

The transmission section 32 transmits the information 502 stored in the memory section 31 to the nitric oxide concentration testing server 4 through the communication network 5. That is, the transmission section 32 transmits the nitric oxide concentration information which is information on a nitric oxide concentration in the breath of a test subject, the measurement time information which is information on the measurement time of a nitric oxide concentration in the breath of a test subject, and the use history information 501 of the drug inhalation device 500 to the nitric oxide concentration testing server 4 including a memory section 13 which stores the nitric oxide concentration information, the measurement time information, and the use history information 501 in association with one another.

According to this embodiment, a chronological relationship between the use time of the drug inhalation device 500 and the measurement time of the nitric oxide concentration can be ascertained, and therefore, it becomes easy to determine which information is highly reliable nitric oxide concentration information. Accordingly, the nitric oxide concentration testing terminal device 3 capable of obtaining highly reliable information can be realized.

Further, the nitric oxide concentration testing terminal device 3 has a similar effect to that of the nitric oxide concentration testing device 1 resulted from a similar configuration to that of the nitric oxide concentration testing device 1 shown in FIG. 2.

### 2-1-3. Configuration of Nitric Oxide Concentration Testing Server

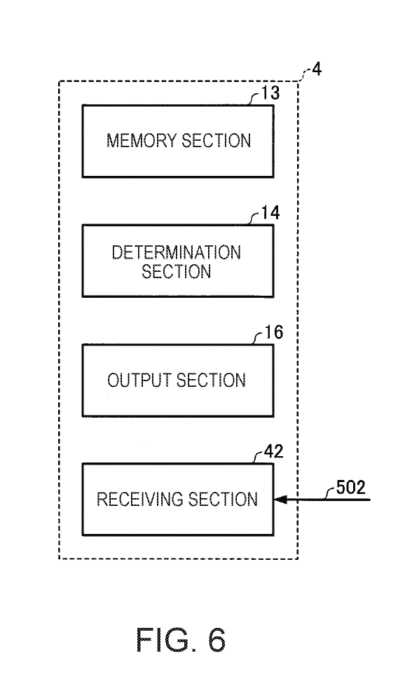

FIG. 6 is a functional block diagram of the nitric oxide concentration testing server 4 according to the first embodiment. The same reference numerals are given to the same components as those in FIG. 2, and a detailed description thereof will be omitted.

In an example shown in FIG. 6, the nitric oxide concentration testing server 4 is configured to include a memory section 13, a determination section 14, an output section 16, and a receiving section 42. Some components shown in FIG. 6 may be omitted, or another component may be added according to a function required for the nitric oxide concentration testing server 4.

The receiving section 42 receives nitric oxide concentration information which is information on a nitric oxide concentration in the breath of a test subject, measurement time information which is information on the measurement time of a nitric oxide concentration in the breath of a test subject, and the use history information 501 of the drug inhalation device 500 from the nitric oxide concentration testing terminal device 3 through the communication network 5.

The memory section 13 stores the nitric oxide concentration information which is information on a nitric oxide concentration in the breath of a test subject, the measurement time information which is information on the measurement time of a nitric oxide concentration in the breath of a test subject, and the use history information 501 which is information on the use history of the drug inhalation device 500 received by the receiving section 42. In this embodiment, the memory section 13 stores the nitric oxide concentration information which is information on a nitric oxide concentration in the breath of a test subject and the measurement time information which is information on the measurement time of a nitric oxide concentration in the breath of a test subject in association with each other. The example of the information stored in the memory section 13 is the same as described with reference to FIG. 3.

According to this embodiment, a chronological relationship between the use time of the drug inhalation device 500 and the measurement time of the nitric oxide concentration can be ascertained, and therefore, it becomes easy to determine which information is highly reliable nitric oxide concentration information. Accordingly, the nitric oxide concentration testing server 4 capable of obtaining highly reliable information can be realized.

Further, the nitric oxide concentration testing server 4 has a similar effect to that of the nitric oxide concentration testing device 1 resulted from a similar configuration to that of the nitric oxide concentration testing device 1 shown in FIG. 2.

### 2-2. Second Embodiment

### 2-2-1. Overall Configuration

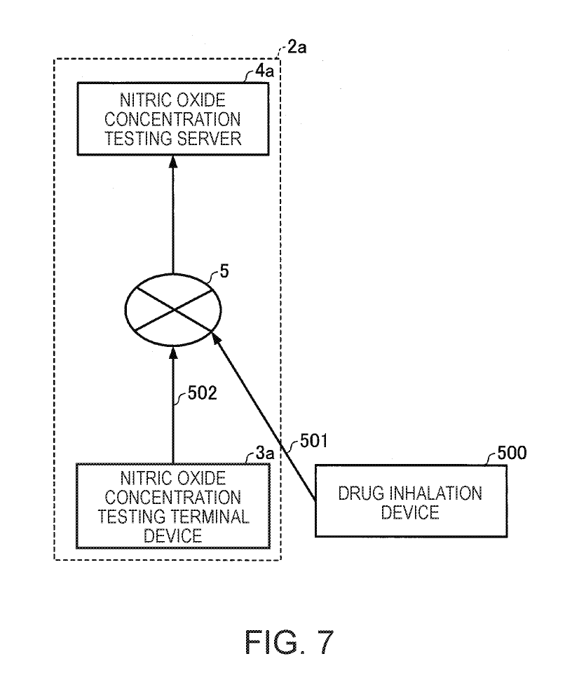

FIG. 7 is a view showing an overall configuration of a nitric oxide concentration testing system 2a according to a second embodiment. The same reference numerals are given to the same components as those in FIG. 4, and a detailed description thereof will be omitted.

The nitric oxide concentration testing system 2a shown in FIG. 7 is configured to include a nitric oxide concentration testing terminal device 3a and a nitric oxide concentration testing server 4a.

The nitric oxide concentration testing server 4a acquires use history information 501 which is information on the use history of a drug inhalation device from a drug inhalation device 500 through a communication network 5. The nitric oxide concentration testing terminal device 3a transmits information 502 (the content of the information 502 will be described later) to the nitric oxide concentration testing server 4a through the communication network 5.

As described below, according to this embodiment, a chronological relationship between the use time of the drug inhalation device 500 and the measurement time of the nitric oxide concentration can be ascertained, and therefore, it becomes easy to determine which information is highly reliable nitric oxide concentration information. Accordingly, the nitric oxide concentration testing system 2a capable of obtaining highly reliable information can be realized.

### 2-2-2. Configuration of Nitric Oxide Concentration Testing Terminal Device

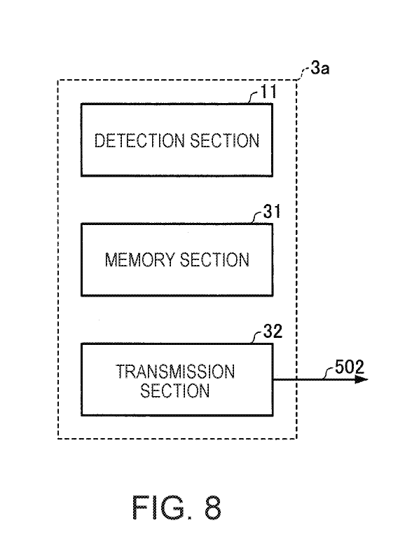

FIG. 8 is a functional block diagram of the nitric oxide concentration testing terminal device 3a according to the second embodiment. The same reference numerals are given to the same components as those in FIG. 5, and a detailed description thereof will be omitted.

In an example shown in FIG. 8, the nitric oxide concentration testing terminal device 3a is configured to include a detection section 11, a memory section 31, and a transmission section 32. Some components shown in FIG. 8 may be omitted, or another component may be added according to a function required for the nitric oxide concentration testing terminal device 3a.

The memory section 31 stores nitric oxide concentration information which is information on a nitric oxide concentration in the breath of a test subject and measurement time information which is information on the measurement time of a nitric oxide concentration in the breath of a test subject in association with each other. The example of the information stored in the memory section 31 is the same as described with reference to FIG. 3.

The transmission section 32 transmits the information 502 stored in the memory section 31 to the nitric oxide concentration testing server 4a through the communication network 5. That is, the transmission section 32 transmits nitric oxide concentration information which is information on a nitric oxide concentration in the breath of a test subject and measurement time information which is information on the measurement time of a nitric oxide concentration in the breath of a test subject to the nitric oxide concentration testing server 4a including a memory section 13 which stores the nitric oxide concentration information, the measurement time information, and the use history information 501 of the drug inhalation device 500 in association with one another.

According to this embodiment, a chronological relationship between the use time of the drug inhalation device 500 and the measurement time of the nitric oxide concentration can be ascertained, and therefore, it becomes easy to determine which information is highly reliable nitric oxide concentration information. Accordingly, the nitric oxide concentration testing terminal device 3a capable of obtaining highly reliable information can be realized.

Further, the nitric oxide concentration testing terminal device 3a has a similar effect to that of the nitric oxide concentration testing device 1 resulted from a similar configuration to that of the nitric oxide concentration testing device 1 shown in FIG. 2.

2-2-3. Configuration of Nitric Oxide Concentration Testing Server

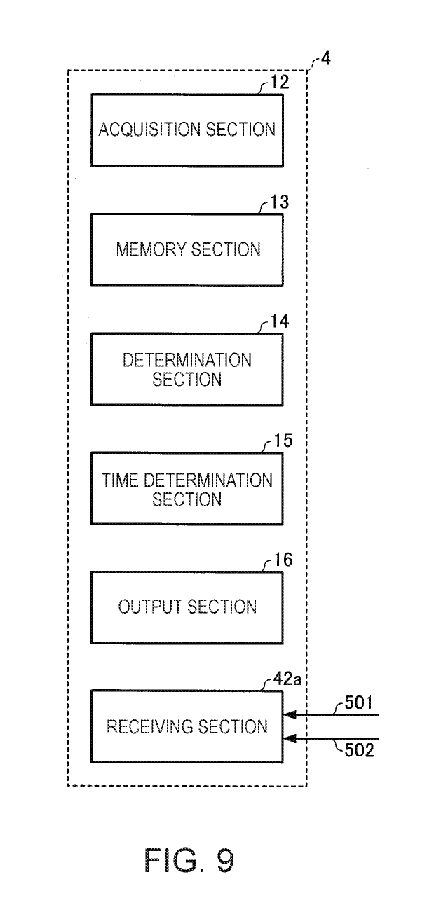

FIG. 9 is a functional block diagram of the nitric oxide concentration testing server 4a according to the second embodiment. The same reference numerals are given to the same components as those in FIG. 6, and a detailed description thereof will be omitted.

In an example shown in FIG. 9, the nitric oxide concentration testing server 4a is configured to include an acquisition section 12, a memory section 13, a determination section 14, a time determination section 15, an output section 16, and a receiving section 42a. Some components shown in FIG. 9 may be omitted, or another component may be added according to a function required for the nitric oxide concentration testing server 4a.

The receiving section 42a receives nitric oxide concentration information which is information on a nitric oxide concentration in the breath of a test subject and measurement time information which is information on the measurement time of a nitric oxide concentration in the breath of a test subject as the information 502 from the nitric oxide concentration testing terminal device 3a through the communication network 5. Further, the receiving section 42a receives the use history information 501 of the drug inhalation device 500 from the drug inhalation device 500 through the communication network 5.

The memory section 13 stores the nitric oxide concentration information which is information on a nitric oxide concentration in the breath of a test subject, the measurement time information which is information on the measurement time of a nitric oxide concentration in the breath of a test subject, and the use history information 501 which is information on the use history of a drug inhalation device 500 received by the receiving section 42a. In this embodiment, the memory section 13 stores the nitric oxide concentration information which is information on a nitric oxide concentration in the breath of a test subject and the measurement time information which is information on the measurement time of a nitric oxide concentration in the breath of a test subject in association with each other. The example of the information stored in the memory section 13 is the same as described with reference to FIG. 3.

According to this embodiment, a chronological relationship between the use time of the drug inhalation device 500 and the measurement time of the nitric oxide concentration can be ascertained, and therefore, it becomes easy to determine which information is highly reliable nitric oxide concentration information. Accordingly, the nitric oxide concentration testing server 4a capable of obtaining highly reliable information can be realized.

Further, the nitric oxide concentration testing server 4a has a similar effect to that of the nitric oxide concentration testing device 1 resulted from a similar configuration to that of the nitric oxide concentration testing device 1 shown in FIG. 2.

### 3. Configuration Example of Detection Section

The detection section 11 according to this embodiment detects the nitric oxide concentration using spectroscopy. More specifically, the detection section 11 detects the nitric oxide concentration using a surface-enhanced Raman scattering method.

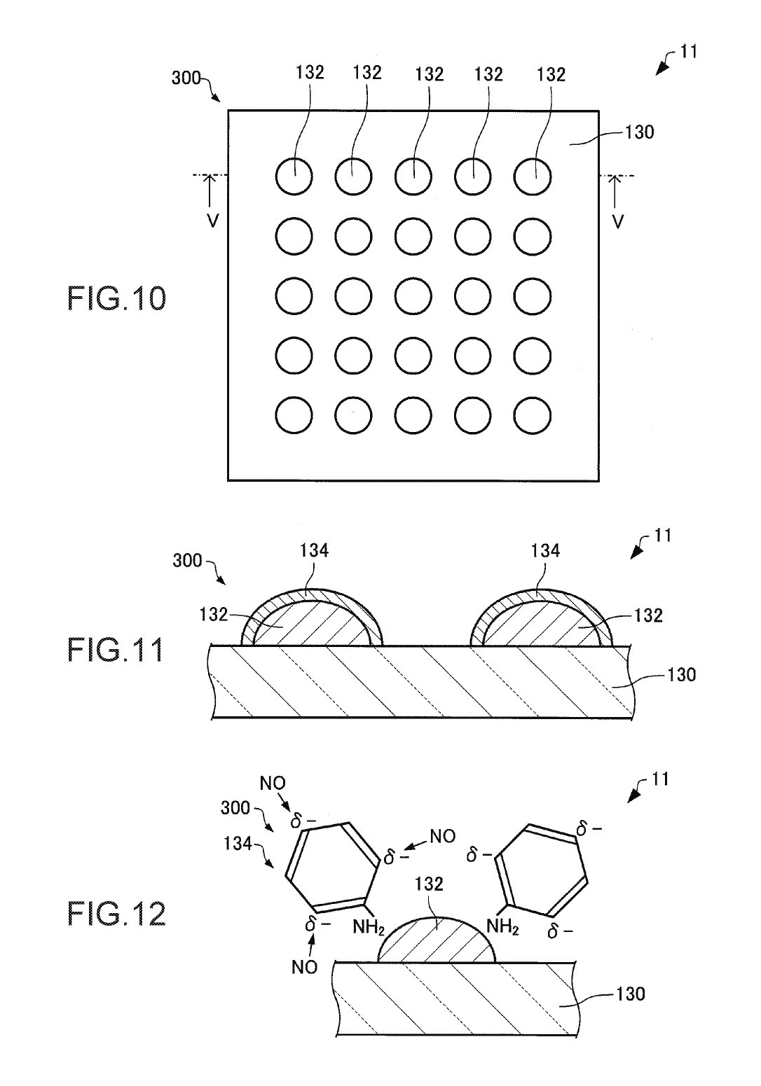

Next, a sensor chip 300 which constitutes part of the detection section 11 will be described. FIG. 10 is a plan view schematically showing the sensor chip 300. FIG. 11 is a cross-sectional view taken along the line V-V in FIG. 10 schematically showing the sensor chip 300. FIG. 12 is a view conceptually showing the principle of trapping nitric oxide.

As shown in FIGS. 10 and 11, the sensor chip 300 includes a substrate 130, a metal microstructure 132, and an organic molecular modification film 134. Incidentally, in FIG. 10, the illustration of the organic molecular modification film 134 is omitted for the sake of convenience.

The substrate 130 is, for example, a glass substrate, a silicon substrate, or a resin substrate.

The metal microstructure 132 is provided on the substrate 130. The shape of the metal microstructure 132 is not particularly limited, and is, for example, a cylinder, a particle, a prism, a sphere, or a spheroid. The size (for example, the diameter) of the metal microstructure 132 is equal to or smaller than the wavelength of light irradiated on the sensor chip 300. Specifically, the size of the metal microstructure 132 is 40 nm or more and 700 nm or less. In the example shown in the drawing, a plurality of metal microstructures 132 are provided. The material of the metal microstructure 132 is, for example, gold, silver, aluminum, or copper. The metal microstructure 132 is formed by, for example, a vacuum deposition method or the like.

When the metal microstructure 132 is irradiated with light, surface plasmon resonance (SPR) occurs. Specifically, in the metal microstructure 132, localized surface plasmon resonance (LSPR) occurs. LSPR is a phenomenon in which when light is incident on a metal structure with a size equal to or smaller than the wavelength of the light, free electrons present in the metal collectively oscillate due to an electric field component of the light, and a local electric field is induced outside. By this local electric field, Raman scattered light can be enhanced. A phenomenon in which Raman scattered light is enhanced by an electric field induced by SPR in this manner is referred to as “electric field enhancement effect”. The intensity of Raman scattered light enhanced by SPR (SERS light) is proportional to the fourth power of the electric field enhanced by SPR.

The organic molecular modification film 134 is provided on the metal microstructure 132. The organic molecular modification film 134 includes a modifying molecule according to this embodiment (hereinafter also simply referred to as “modifying molecule”). The modifying molecule is disposed on the surface of the metal microstructure 132. The organic molecular modification film 134 is formed by, for example, immersing the substrate 130 having the metal microstructure 132 formed thereon in a solution in which the modifying molecule is diluted (for example, at a concentration of 1 mM) for a long time (for example, 24 hours), and thereafter, taking out the substrate 130 from the solution and blowing off water.

The modifying molecule is derived from, for example, a compound having an amine-based functional group. Specifically, the modifying molecule is derived from aniline which is a benzene ring having an amine group (see the following formula (1)). In this case, the modifying molecule binds to the metal microstructure 132 through the amine group as shown in FIG. 12. It is considered that NO (nitrogen monoxide) reacts with oxygen in the air and is converted to N₂O₃ as a reactant, and N₂O₃ having an electron-withdrawing property reacts with the benzene ring at an electron localization site (a site denoted by δ- shown in FIG. 12). In this manner, the sensor chip 300 can trap NO.

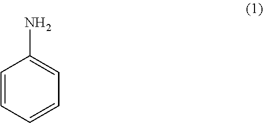

The modifying molecule may be derived from a compound which has a sulfur-based functional group instead of having an amine-based functional group. Specifically, the modifying molecule may be derived from methanethiol (CH₃SH). Here, the phrase “derived from a given compound (for example, aniline or methanethiol)” refers to that the compound is bound to the metal microstructure 132 through a bond such as a coordinate bond, a covalent bond, an ionic bond, or a hydrogen bond as it is or by detaching part of a substituent, whereby the modifying molecule is obtained.

According to this embodiment, the nitric oxide concentration is detected using spectroscopy, and therefore, the nitric oxide concentration testing device 1, the nitric oxide concentration testing system 2, the nitric oxide concentration testing terminal device 3, the nitric oxide concentration testing system 2a, and the nitric oxide concentration testing terminal device 3a, each of which has excellent measurement reliability, can be realized.

Further, according to this embodiment, the nitric oxide concentration is detected using a surface-enhanced Raman scattering method, and therefore, the nitric oxide concentration testing device 1, the nitric oxide concentration testing system 2, the nitric oxide concentration testing terminal device 3, the nitric oxide concentration testing system 2a, and the nitric oxide concentration testing terminal device 3a, each of which is less likely to be affected by the environment, and is highly sensitive and small in size can be realized.

Hereinabove, the embodiments and modifications have been described, however, the invention is not limited thereto and can be implemented in various modes without departing from the gist of the invention.

The invention includes substantially the same configurations (for example, configurations having the same function, method, and result, or configurations having the same object and effect) as the configuration described in the embodiment. Further, the invention includes configurations in which a nonessential part of the configuration described in the embodiment is replaced. In addition, the invention includes configurations having the same operational effect as that of the configuration described in the embodiment, or configurations capable of achieving the same object. Moreover, the invention includes configurations in which a known art is added to the configuration described in the embodiment.

The entire disclosure of Japanese Patent Application No. 2015-184811, filed Sep. 18, 2015 is expressly incorporated by reference herein.

What is claimed is:

1. A nitric oxide concentration testing device, comprising:
a detection section which detects a nitric oxide concentration in the breath of a test subject;
an acquisition section which acquires use history information which is information on the use history of a drug inhalation device; and
a memory section which stores nitric oxide concentration information which is information on the nitric oxide concentration, measurement time information which is information on the measurement time of the nitric oxide concentration, and the use history information.

2. The nitric oxide concentration testing device according to claim 1, wherein the device includes a determination section which determines the validity of the nitric oxide concentration information based on information on the use time of the drug inhalation device obtained from the use history information and the measurement time information.

3. The nitric oxide concentration testing device according to claim 1, wherein the device includes a time determination section which determines the use time of the drug inhalation device based on the use history information.

4. The nitric oxide concentration testing device according to claim 3, wherein the use history information includes information on the movement of the drug inhalation device.

5. The nitric oxide concentration testing device according to claim 3, wherein the use history information includes information on the operation of the drug inhalation device.

6. The nitric oxide concentration testing device according to claim 1, wherein the detection section detects the nitric oxide concentration using spectroscopy.

7. The nitric oxide concentration testing device according to claim 6, wherein the detection section detects the nitric oxide concentration using a surface-enhanced Raman scattering method.

8. A nitric oxide concentration testing system, comprising:
a detection section which detects a nitric oxide concentration in the breath of a test subject;
an acquisition section which acquires use history information which is information on the use history of a drug inhalation device; and
a memory section which stores nitric oxide concentration information which is information on the nitric oxide concentration, measurement time information which is information on the measurement time of the nitric oxide concentration, and the use history information in association with one another.

9. A nitric oxide concentration testing server, comprising:
a receiving section which receives nitric oxide concentration information which is information on a nitric oxide concentration in the breath of a test subject, measurement time information which is information on the measurement time of the nitric oxide concentration, and use history information which is information on the use history of a drug inhalation device from a nitric oxide concentration testing terminal including a detection section which detects the nitric oxide concentration and an acquisition section which acquires the use history information; and
a memory section which stores the nitric oxide concentration information, the measurement time information, and the use history information.

10. A nitric oxide concentration testing terminal device, comprising:
a detection section which detects a nitric oxide concentration in the breath of a test subject;
an acquisition section which acquires use history information which is information on the use history of a drug inhalation device; and
a transmission section which transmits nitric oxide concentration information which is information on the nitric oxide concentration, measurement time information which is information on the measurement time of the nitric oxide concentration, and the use history information to a nitric oxide concentration testing server including a memory section which stores the nitric oxide concentration information, the measurement time information, and the use history information in association with one another.

11. A nitric oxide concentration testing server, comprising:
a receiving section which receives nitric oxide concentration information which is information on a nitric oxide concentration in the breath of a test subject and measurement time information which is information on the measurement time of the nitric oxide concentration from a nitric oxide concentration testing terminal including a detection section which detects the nitric oxide concentration;
an acquisition section which acquires use history information which is information on the use history of a drug inhalation device; and
a memory section which stores the nitric oxide concentration information, the measurement time information, and the use history information.

12. A nitric oxide concentration testing terminal device, comprising:
a detection section which detects a nitric oxide concentration in the breath of a test subject; and
a transmission section which transmits nitric oxide concentration information which is information on the nitric oxide concentration and measurement time information which is information on the measurement time of the nitric oxide concentration to a nitric oxide concentration testing server including a memory section which stores the nitric oxide concentration information, the measurement time information, and use history information which is information on the use history of a drug inhalation device in association with one another.
